(19) United States
(12) Patent Application Publication
(10) Pub. No.: US 2004/0133129 A1
(43) Pub. Date: Jul. 8, 2004

(54) Measurements in a body lumen using guidewire with spaced markers

(75) Inventors: Eran Harari, Doar Na Menashe (IL); Avraham Rapaport, Tel Aviv (IL); Yaron Asaf, Doar Na Menashe (IL)

Correspondence Address: G.E. EHRLICH (1995) LTD., c/o ANTHONY CASTORINA, SUITE 207, 2001 JEFFERSON DAVIS HIGHWAY, ARLINGTON, VA, 22202, US

(73) Assignee: MindGuard Ltd.

(21) Appl. No.: 10/335,998

(22) Filed: Jan. 3, 2003

(51) Int. Cl. A61B 5/00

(52) U.S. Cl. 600/585

(57) ABSTRACT

A method is provided for measuring a distance between a first location and a second location within a body lumen. The method includes providing a guidewire having radiopaque elements of predetermined dimensions and spacings, inserting the guidewire into the body lumen, imaging the guidewire as it progresses through the body lumen, positioning the radiopaque markers in the vicinity of the first and second locations, and determining a distance from the predetermined dimensions and spacings.

### FIELD AND BACKGROUND OF THE INVENTION

The present invention relates to a method for measurement within a body lumen and, more particularly, to a method for measuring parameters within a body lumen using a guidewire with spaced markers.

In the medical diagnostic field, it is often useful to measure a body lumen or a segment thereof. For example, during certain procedures such as balloon angioplasty or graft or stent placement, it is important to be able to accurately determine distances within blood vessels for positioning of grafts or stents, and diameters of blood vessels for choosing appropriately sized devices for insertion, or to be able to accurately measure an area of a lesion within the blood vessel.

These types of measurements are often obtained using fluoroscopy. In fluoroscopy, a contrast agent is injected into a vessel, and the vessel is then imaged radiographically. A disadvantage of the fluoroscopy method is that only a planar view (two-dimensional) is produced. As a result, angiograms often fail to reveal the presence of winding paths of the examined vessel, which may not progress along the plane of the angiogram. Hence, length measurements are not always accurate since the measurement method does not account for the fact that the vessel does not necessarily lie in the same plane as the image. Furthermore, the vessels are generally curved, and not in a straight line, which can further distort the measurements.

Another method used for measurement inside a body lumen such as a blood vessel is Computerized Tomography (CT) scanning. CT scans depict blood vessel diameters from which other desired measurements, such as length, can be extrapolated. However, the prediction of length based solely upon slices of diameter limit the accuracy of CT scans. Moreover, CT scanning systems are rarely used since they are expensive and do not provide real time measurements.

A more advanced method is intravascular ultrasound (IVUS), in which an array of transducers located around a tip of a catheter is inserted into a blood vessel. An ultrasound beam is rotated within the blood vessel, forming a 360-degree cross-sectional image. Similar to the CT scanning method, the IVUS method does not provide a direct length measurement. Furthermore, lengths and diameters are often presented using different measurements using standard measurement tools. For example, length is usually presented in millimeters or centimeters, while diameter is presented in units of French. Another disadvantage shared by the CT scanning and IVUS, is that they only provide instantaneous views of the vessel, and may therefore not be accurately representative of the vessel diameter during systole or diastole of the vessel.

There is thus a great need for and it would be highly advantageous to have a method for directly providing measurements within a body lumen, such as a blood vessel, devoid of the above limitations.

### SUMMARY OF THE INVENTION

According to one aspect of the present invention there is provided a method of measurement within a body lumen, the method including providing a guidewire having radiopaque elements spaced apart at predetermined distances, inserting the guidewire into a body lumen, imaging the guidewire having the radiopaque elements as it progresses through the body lumen, and determining a dimensional property from the predetermined distances.

According to another aspect of the present invention there is provided a method for measuring a distance between a first location and a second location within a body lumen, the method including positioning a first radiopaque marker located on a guidewire generally adjacent to the first location, positioning a second radiopaque marker located on a guidewire generally adjacent to the second location, visualizing the first and second radiopaque markers, and measuring the distance between the first and second radiopaque markers.

According to yet another aspect of the present invention there is provided a method for measuring a dimensional property between a first location and a second location within a body lumen, the method including providing a guidewire having radiopaque elements spaced apart at predetermined distances, inserting the guidewire into a body lumen, imaging the guidewire as it progresses through the body lumen, positioning the radiopaque markers in the vicinity of the first and second locations, and determining the property from the predetermined distances.

According to yet another aspect of the present invention there is provided a method for simultaneously measuring a length and a diameter of a portion of a body lumen, the method including providing a guidewire having radiopaque elements spaced apart at predetermined distances, the radiopaque elements having specific dimensions, inserting the guidewire into the body lumen, imaging the guidewire as it progresses through the body lumen, positioning the guidewire at the portion of the body lumen, and determining the length and the diameter from the predetermined distances and from the specific dimensions of the radiopaque elements. The length and diameter may be measured in the same units or in different units.

According to yet another aspect of the present invention there is provided a method of measurement within a body lumen, the method including providing a guidewire having radiopaque elements spaced apart at predetermined distances, the radiopaque elements having specific dimensions, inserting the guidewire into the body lumen, imaging the guidewire having the radiopaque elements as it progresses through the body lumen, and determining a dimensional property from the predetermined distances and from the specific dimensions of the radiopaque marker.

According to further features in preferred embodiments of the invention described below, the body lumen is a blood vessel, and the imaging is done using fluoroscopy and may include labeling of the radiopaque markers.

According to still further features in the described preferred embodiments, the dimensional property is a length of a portion of a blood vessel or of an occlusion. According to other embodiments, the dimensional property is a depth, a curvature, a diameter, an angle, or a three-dimensional projection of the body lumen.

According to further features in other embodiments of the invention described below, the predetermined distances are evenly spaced, or vary according to some predetermined formula.

According to further features in other embodiments of the invention described below, the determining is done by counting the radiopaque elements on a monitor, by observing spaces between the radiopaque elements as reflected on the monitor, or by counting differences in spacing as reflected on the monitor and as measured on the guidewire.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a diagrammatic view showing a blood vessel that has been occluded with deposits along an inner wall and shows the positioning of a flexible guidewire within the blood vessel, and a monitor for viewing the position;

FIG. 2 is a planar view of a guidewire showing markers and spacings, in accordance with one embodiment of the present invention;

FIG. 3 is a an elevation-segmented view of a flexible guidewire core wire constructed in accordance with one embodiment of the present invention; and

FIG. 4 is a section view of a segment of a guidewire, constructed in accordance with another embodiment of the present invention.

### DESCRIPTION OF THE PREFERRED EMBODIMENTS

The present invention is of a method of measurement within a body lumen using a guidewire with radiopaque elements, the elements having specific dimensions and predetermined spacing.

The principles and operation of the method of the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

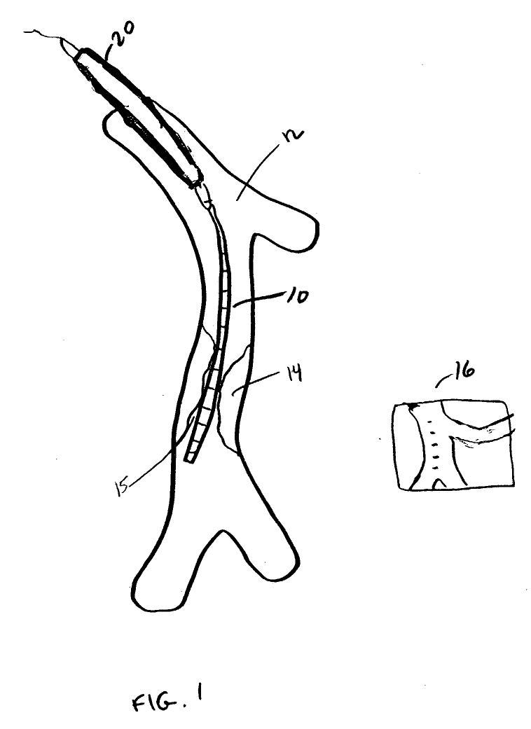

Turning now to the drawings, FIG. 1 illustrates a flexible, small diameter guidewire 10 that can be guided through a patient cardiovascular system. A distal end of the guidewire 10 is shown in FIG. 1 approaching a region in a blood vessel 12 having occlusions 14 which restrict blood flow through the blood vessel 12. As the guidewire 10 is inserted along the winding path to the obstructed blood vessel region, a user (for example, an attending physician) conducting the procedure monitors progress of the guidewire 10 on a monitor 16.

The guidewire 10 is long enough to be routed from a patient entry point through the patient to the obstructed blood vessel region. In a preferred embodiment the guidewire 10 has a length L of 260 cm and a diameter of 0.35″. In other embodiments, the guidewire 10 has a length L of 100 cm to 300 cm and a diameter of 0.012″ to 0.38″.

In a preferred embodiment, the guidewire is used for routing a catheter 20 to the vicinity of occlusion 14. In one embodiment of the present invention, catheter 20 is a balloon catheter, in which a balloon may be deployed within the vicinity of the occlusion 14 in order to compress the deposits that have accumulated along the inner walls of, for example, an artery, thus widening the artery lumen and increasing blood flow. A balloon expandable stent may be deployed on the balloon catheter for deployment within the walls of the blood vessel to compress the deposits in the area of the obstruction or occlusion and to provide further structural support. In another embodiment of the present invention, catheter 20 may further comprise a self-expandable stent, in which no balloon is necessary for deployment of the stent.

In use, a distal end of the guidewire 10 is routed through a narrow passageway 15 in occlusion 14, and the catheter 20 is placed over the guidewire 10 until the balloon and/or stent bridges the region of occlusion 14 within the blood vessel 12. The balloon and/or stent is then expanded and the outer surface contacts occlusion 14. The inner walls of occlusion 14 are compressed and a wider lumen or passageway is created in the blood vessel 12.

During positioning of the guidewire 10 within the cardiovascular system, it is desirable to be able to view the progress and locate the guidewire within the body. The procedure is widely performed under fluoroscopy, in which X-ray radiation is used to visualize radiopaque segments of the guidewire. Thus, a physician or other user is able to watch the radiopaque segments on monitor 16 as the procedure progresses. Once the guidewire 10 is situated in place, the catheter 20 is advanced.

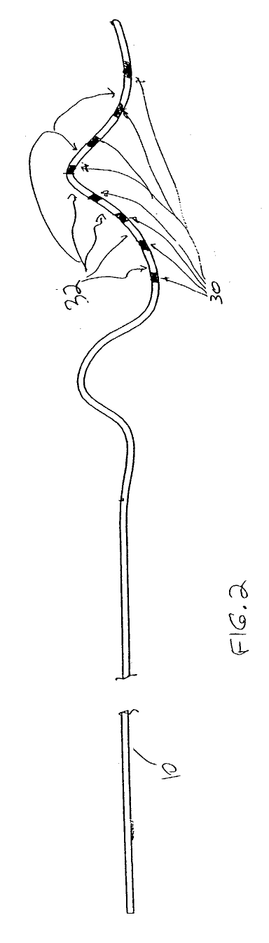

As described in detail below, the guidewire 10 is constructed so that bands 30 or regions of high radiopacity appear when the blood vessel 12 is monitored on a viewing screen. As shown in FIG. 2, the bands 30 are separated at predetermined distances 32 thereby giving a reference length. In one embodiment, the distances 32 are spaced evenly. In another embodiment, varying distances 32 may be used. Furthermore, the radiopaque bands 30 themselves have measurable dimensions. In a preferred embodiment, the distance 32 between markers is between 5 and 15 mm. In a preferred embodiment, the radiopaque markers or bands 30 have lengths between 1 and 8 French, preferably 1, 2, 3, 5, 6 or 8 French.

The opacity of the bands 30 can be varied as well, such that, for example, the opacity diminishes at the distal or working end of the guidewire 10. This would allow adequate tracing of the guidewire 10 while minimizing interference with a post procedure angiogram.

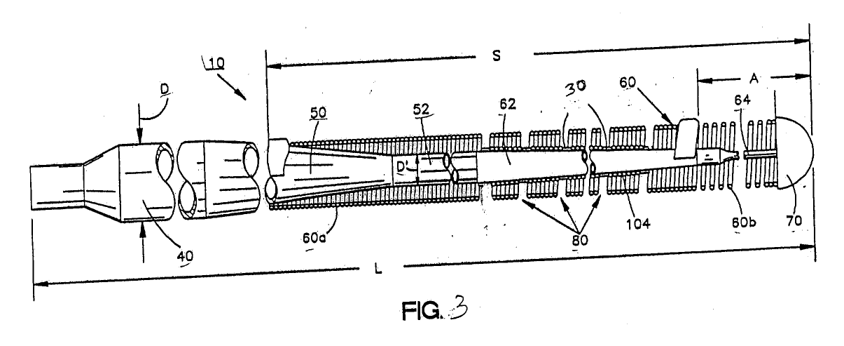

It should be readily apparent that many possibilities exist for the construction of guidewire 10. For example, guidewire 10 may be a commercially available guidewire such as the “Magic Marker” from Boston Scientific (Reference number 46-592). Alternatively, guidewire 10 may be constructed similar to one described in U.S. Pat. No. 5,353,808 to Viera, incorporated herein by reference in its entirety, and illustrated in FIG. 3.

Turning now to FIG. 3, the guidewire 10 is seen to include a center stainless steel or other suitable flexible wire core 40 having a first uniform diameter D, in the range of 0.0100-0.038 inches, extending well over half the length “L” of the guidewire 10. To improve the depiction of details of the distal portion of the guidewire 10, this uniform diameter elongated portion has been sectioned and a major portion of its length deleted from FIG. 3.

The total length of the uniform diameter portion 40 is approximately 110 to 270 cm of the total guidewire length of 100 to 300 cm. It is typically covered with a suitable coating to make its outer surface lubricious. A short proximal portion of the core 40 is exposed. The remaining distal segment of the guidewire 10 has a length S of approximately 27 cm.

At the guide wire's distal end, the wire core 40 tapers along a portion 50 uniformly to a portion 52 having a uniform diameter D′. A coiled wire spring 60 covers a distal portion of the core wire. A first portion 60a of the spring 60 is constructed of a low radiopaque wire having a thickness of 0.0025-0.004 inches and is attached to the tapered center core portion 50. The core 40 again tapers uniformly along a segment 62. An extreme distal segment 64 of the core 40 is flattened and surrounded by a second less tightly coiled portion 60b of the spring 60 constructed from a radiopaque wire having the same thickness as the wire that forms the first portion 60a. This distal segment of the guidewire 10 has a length A of approximately 1 inch and can be pre-bent to a particular configuration by the attending physician to facilitate insertion of the guidewire within the subject.

At the extreme distal tip portion of the guidewire 10, a weld 70 attaches the distal portion 60b of the spring 60 to the flattened portion 64 of the core. The weld defines a smooth hemispherical bead, which does not damage the inner lining of the blood vessels as the guidewire tip comes in contact with those linings.

The spring 60 is closely packed along the tapered core portion 50 and uniform diameter portion 52 so that adjacent coils of the spring 60 touch each other. The coils of the spring portion 60a are less tightly packed at fixed distances to define gaps or spaces 80. These spaces 80 overlie multiple high radiopaque bands 30 or rings separated by stainless steel coil segments 104. The bands 30 are tungsten, gold, platinum, or any other radiopaque material, and are spaced apart a fixed distance and provide a length reference for a physician viewing the core wire 10 on a viewing screen. Each band 30 on the core wire corresponds to a band 30 on the viewing screen depicted in FIG. 1.

The spaces 80 between bands 30 can be adjusted depending upon the intended use of the guidewire. In a preferred embodiment, the spacings are the same between adjacent bands 30 and are determined by the number of coils in the coil segments 104. While several adjacent coils are depicted between adjacent bands 30 in FIG. 2, it is appreciated that many more coils would be used in fabricating the guidewire to achieve band spacing of approximately one-half inch. If it is desired to have the shades of the visible bands 30 lighter, a different alloy or material is utilized for different bands 30.

In further embodiments of the present invention, guidewire 10 may be constructed of any biocompatible material. For example, guidewire 10 may be constructed of stainless steel, titanium, or any other biocompatible metal. Alternatively, guidewire 10 may be constructed of memory shaped alloys, such as Nitinol. In an alternative embodiment, guidewire 10 may be constructed of a polymer such as PTFE, polylactide, polyglycolide, nylon, or any other biocompatible polymer known in the art.

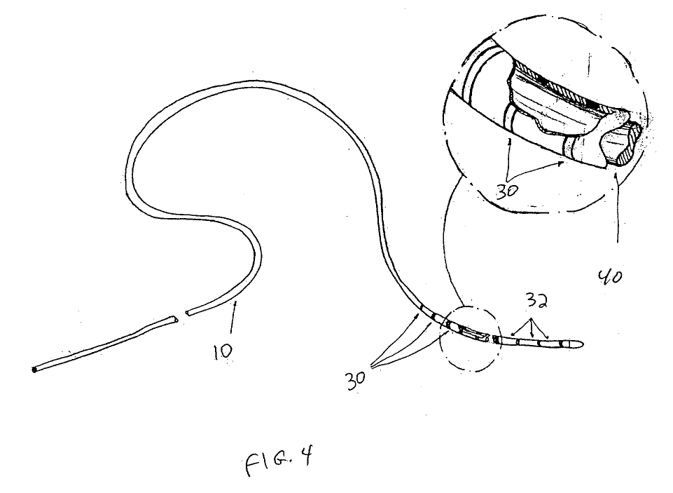

Reference is now made to FIG. 4, which shows a construction of guidewire 10 according to yet another embodiment of the present invention. As shown in FIG. 4, a composite is used, wherein the core 40 is fabricated from a different material than the outer portion of guidewire 10. For example, the outer portion may be constructed of a metal and core 40 may be constructed of a polymer. Alternatively, both portions may be of similar material.

In a further embodiment of the present invention, the radiopaque markers are labeled so that a viewer can immediately determine a length measurement without having to count markers. In this way, measurements are more accurate since any potentially hidden markers in the image will not affect the reading. One way of labeling the markers is to include an additional piece for each additional marker. For example, the first marker includes one radiographic strip of material; the second marker includes two radiographic strips of material, etc. Alternatively, the first marker has a first length; the second marker has a second length, etc.

By using a guidewire such as the one described above, it is possible to correct for the problems associated with taking measurements inside a body lumen. Since the bands 30 are spaced within predetermined intervals, it essentially acts as a ruler. Thus, the blood vessel can be measured, and distances can be obtained without distortion due to curvature of the blood vessel. In addition, no complicated calculations are necessary. Furthermore, since the disclosed method corrects for distortions in viewing of the blood vessels, a physician can be assisted in positioning of the wire accurately, with fewer trials and consequently less trauma to the patient.

In a preferred embodiment, a guidewire such as the one described above is inserted into a blood vessel or other body lumen. As it progresses through the vessel, it is viewed under fluoroscopy on a monitor. At any given point, the viewer can immediately see distances between points within the lumen. By having predetermined spacings between and dimensions of the radiopaque markers, lengths and diameters of the lumen, or lengths of occlusions may be determined.

In addition to simple length measurements, other measurements such as diameter of the lumen, curvature of the vessel, and angles relating to the vessel can be determined. Furthermore, a three-dimensional projection of the lumen can be mapped from the markers as viewed on the screen. If a comparison is made between the spacings between markers as observed on the monitor and as measured on the guidewire, it is possible to map out various parameters related to curvature, three-dimensional projection, and the like. For example, it is possible to measure the diameter of a body lumen by using an arbitrary scale or ruler, calibrating it by comparing it to the guide wire on the angiogram, then turning the pixel 90° and directly measuring the diameter. This can also be done manually or electronically.

It should be noted that encompassed within the scope of the invention is any guidewire, which can be inserted into a body lumen, for example, a guidewire that can be inserted into the digestive tract for use with an endoscope.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A method of measurement within a body lumen, the method comprising:
providing a guidewire having radiopaque elements spaced apart at predetermined distances;
inserting said guidewire into said body lumen;
imaging said guidewire having said radiopaque elements as it progresses through said body lumen; and
determining a dimensional property from said predetermined distances.

2. The method of claim 1, wherein said body lumen is blood vessel.

3. The method of claim 1, wherein said dimensional property is a length.

4. The method of claim 3, wherein said length is that of a portion of a blood vessel.

5. The method of claim 3, wherein said length is that of an occlusion within a blood vessel.

6. The method of claim 1, wherein said dimensional property is a diameter.

7. The method of claim 1, wherein said dimensional property is a curvature.

8. The method of claim 1, wherein said dimensional property is an angle.

9. The method of claim 1, wherein said dimensional property relates to a three-dimensional projection of said body lumen.

10. The method of claim 1, wherein said dimensional property is a diameter.

11. The method of claim 1, wherein said predetermined distances are evenly spaced.

12. The method of claim 1, wherein said predetermined distances vary.

13. The method of claim 1, wherein said determining is done by counting said radiopaque elements on a monitor.

14. The method of claim 1, wherein said determining is done by observing spaces between said radiopaque elements as reflected on said monitor.

15. The method of claim 1, wherein said determining is done by counting differences in spacing as reflected on said monitor and as measured on said guidewire.

16. The method of claim 1, wherein said imaging is done using fluoroscopy.

17. The method of claim 1, further comprising labeling said radiopaque markers.

18. A method for measuring a distance between a first location and a second location within a body lumen, said method comprising:
positioning a first radiopaque marker located on a guidewire generally adjacent to the first location;
positioning a second radiopaque marker located on a guidewire generally adjacent to the second location;
visualizing said first and second radiopaque markers; and
measuring the distance between said first and second radiopaque markers.

19. The method of claim 18, wherein the body lumen is a blood vessel.

20. The method of claim 18, wherein said visualizing is done using fluoroscopy.

21. A method for measuring a dimensional property between a first location and a second location within a body lumen, said method comprising:
providing a guidewire having radiopaque elements spaced apart at predetermined distances;
inserting said guidewire into said body lumen;
imaging said guidewire as it progresses through said body lumen;
positioning said radiopaque markers in the vicinity of the first and second locations; and
determining the property from the predetermined distances.

22. The method of claim 21, wherein the body lumen is a blood vessel.

23. The method of claim 21, wherein the imaging is done using fluoroscopy.

24. The method of claim 21, wherein the property is distance.

25. The method of claim 24, wherein said determining is done by counting said radiopaque markers.

26. The method of claim 21, wherein the property is curvature.

27. The method of claim 21, wherein the property is an angle.

28. The method of claim 21, wherein the property is depth.

29. The method of claim 21, wherein the property is related to a three dimensional projection of said body lumen.

30. The method of claim 21, wherein the property is a diameter.

31. The method of claim 21, wherein said determining is done by counting spaces between said radiopaque elements as reflected on a monitor.

32. The method of claim 21, wherein said determining is done by observing differences in spacing as reflected on a monitor and as measured on said guidewire.

33. The method of claim 21, wherein said radiopaque elements are labeled.

34. The method of claim 26, wherein said determining is done by observing said radiopaque markers on a monitor.

35. A method for simultaneously measuring a length and a diameter of a portion of a body lumen, the method comprising:
providing a guidewire having radiopaque elements spaced apart at predetermined distances, said radiopaque elements having specific dimensions;
inserting said guidewire into said body lumen;
imaging said guidewire as it progresses through said body lumen;
positioning said guidewire at the portion of the body lumen; and
determining the length and the diameter from the predetermined distances and from the specific dimensions of said radiopaque elements.

36. A method as in claim 35, wherein the body lumen is a blood vessel.

37. A method as in claim 35, wherein the length and diameter are measured in the same units.

38. A method as in claim 35, wherein the length and diameter are measured in different units.

39. A method as in claim 35, wherein the predetermined distances are evenly spaced.

40. A method as in claim 35, wherein the predetermined distances vary.

41. A method of measurement within a body lumen, the method comprising:
providing a guidewire having radiopaque elements spaced apart at predetermined distances, said radiopaque elements having specific dimensions;
inserting said guidewire into said body lumen;
imaging said guidewire having said radiopaque elements as it progresses through said body lumen; and
determining a dimensional property from said predetermined distances and from said specific dimensions of said radiopaque marker.

42. The method of claim 41, wherein said body lumen is blood vessel.

43. The method of claim 41, wherein said dimensional property is a length.

44. The method of claim 43, wherein said length is that of a portion of a blood vessel.

45. The method of claim 43, wherein said length is that of an occlusion within a blood vessel.

46. The method of claim 41, wherein said dimensional property is a depth.

47. The method of claim 41, wherein said dimensional property is a curvature.

48. The method of claim 41, wherein said dimensional property is an angle.

49. The method of claim 41, wherein said dimensional property relates to a three-dimensional projection of said body lumen.

50. The method of claim 41, wherein said dimensional property is a diameter.

51. The method of claim 41, wherein said predetermined distances are evenly spaced.

52. The method of claim 41, wherein said predetermined distances vary.

53. The method of claim 41, wherein said determining is done by counting said radiopaque elements on a monitor.

54. The method of claim 41, wherein said determining is done by observing spaces between said radiopaque elements as reflected on said monitor.

55. The method of claim 41, wherein said determining is done by counting differences in spacing as reflected on said monitor and as measured on said guidewire.

56. The method of claim 41, wherein said determining is done by observing the spacing between and the dimensions of said radiopaque markers.

57. The method of claim 41, wherein said imaging is done using fluoroscopy.

58. The method of claim 41, further comprising labeling said radiopaque markers.
